(19) United States
(12) Patent Application Publication
(10) Pub. No.: US 2024/0002982 A1
(43) Pub. Date: Jan. 4, 2024

(54) SUPERELASTIC ALLOYS

(71) Applicant: Royal Melbourne Institute of Technology, Melbourne, Victoria (AU)

(72) Inventors: Ali RAMEZANNEJAD, Melbourne, Victoria (AU); Ma QIAN, Melbourne, Victoria (AU); Wei XU, Melbourne, Victoria (AU)

(21) Appl. No.: 18/252,795

(22) PCT Filed: Nov. 12, 2021
(86) PCT No.: PCT/AU2021/051346

§ 371 Date: May 12, 2023

(30) Foreign Application Priority Data
Nov. 13, 2020 (AU) 2020904162

(51) Int. Cl. C22C 14/00; C22C 1/02; C22F 1/00; C22F 1/18; A61C 7/20; A61L 31/02; A61L 31/14

(52) U.S. Cl. CPC C22C 14/00; C22C 1/02; C22F 1/002; C22F 1/183; A61C 7/20; A61L 31/022; A61L 31/14; A61C 2201/007; A61L 2400/16

(57) ABSTRACT

Disclosed herein is a superelastic alloy comprising tin, in an amount of between 1 at. % and 8 at. %, niobium, in an amount of between 1 at. % and 10 at. % and iron, in an amount of between 0.5 at. % and 3 at. %. The alloy may also optionally comprise oxygen, in an amount of between 0 and 2 at. % and zirconium, in an amount of between 0 and 10 at. %. The balance of the alloy composition is titanium and unavoidable impurities.

### TECHNICAL FIELD

The present invention relates to titanium-based and nickel-free superelastic alloys.

### BACKGROUND ART

Superelastic materials are very useful for a number of applications. When mechanically loaded, superelastic materials deform reversibly to relatively high strains via a reversible stress-induced transformation from the parent (unloaded) phase to a metastable product phase. When the load is removed, however, the stress-induced product phase becomes unstable and transforms back to the parent phase, whereupon the material returns to its original shape. Thus, superelastic materials only undergo plastic deformation when subject to significantly higher applied strains compared to that of non-superelastic materials. Superelastic materials can therefore accommodate much greater strain than non-superelastic materials, and ultimately recover their shape upon release of the mechanical loading.

Nickel titanium superelastic alloys (generally referred to herein as “Superelastic NiTi”) have been commercially available as a product known as NITINOL since the early 1970s. NITINOL alloys contain roughly equiatomic amounts of titanium and nickel and exhibit excellent superelasticity, as quantified by a maximum recoverable strain of up to 8%. NITINOL alloys have been widely used in dental applications such as orthodontic archwires, and in medical appliances such as cardiovascular stents, heart valve frames, orthopaedic staples and single-use suture passers.

In recent times, however, nickel toxicity has become a concern, with nickel-containing body implants being found to release nickel ions when in contact with the body fluids, causing health issues (due to the cytotoxicity and allergenic properties of nickel). There has therefore been a concerted effort to develop new titanium-containing alloys that have superelastic properties, but which do not contain nickel (or other potentially bio-incompatible elements). However, whilst a number of such alloys have been found, none exhibit superelasticity comparable to that of the NITINOL alloys. Furthermore, no nickel-free titanium-based superelastic alloys have been commercially successful to date, due to problems such as cost and reliable manufacture.

It would be advantageous to provide alternative titanium-based, nickel-free alloys which are biocompatible and which exhibit superelastic properties.

### SUMMARY OF INVENTION

In a first aspect, the present invention provides a superelastic alloy comprising tin, in an amount of between 1 at. % and 8 at. %, niobium, in an amount of between 1 at. % and 10 at. % and iron, in an amount of between 0.5 at. % and 3 at. %. The alloy may also optionally comprise oxygen, in an amount of between 0 and 2 at. % and zirconium, in an amount of between 0 and 10 at. %. The balance of the alloy composition is titanium and unavoidable impurities.

Advantageously, the present invention provides a superelastic alloy which, as will be described in further detail below, includes embodiments that surprisingly have a superelasticity that is expected to be comparable with that of the industry leading NITINOL alloys (but without containing Ni) and which is better than any of the presently known nickel-free titanium-based superelastic alloys. Furthermore, the alloys of the present invention utilise relatively inexpensive elemental components, and which have relatively low melting temperatures, thereby potentially simplifying their manufacture.

In some embodiments, the alloy may comprise between 4-6 at. % tin. In some embodiments, the alloy may comprise between 1-4 at. % niobium. In some embodiments, the alloy may comprise between 2-3 at. % iron. Specific superelastic alloys in accordance with embodiments of the present invention and which are described in detail below are: Ti-2.5Nb-2.5Fe-4Sn (at. %), Ti-2.5Nb-2.5Fe-5Sn (at. %) and Ti-2.5Nb-2.5Fe-6Sn (at. %).

In some embodiments, the alloy may comprise between 0.5-1.5 at. % oxygen. Advantageously, including oxygen in the alloy composition may enable the use of small quantities of titanium oxide or less pure forms of elemental titanium instead of pure elemental titanium when manufacturing the alloy, which would even further reduce its cost of manufacture.

In a second aspect, the present invention provides a superelastic alloy consisting essentially of tin, niobium, iron, titanium and, optionally, oxygen and/or zirconium, the alloy having a metastable β-phase microstructure at human body temperature and exhibiting a β to α″-phase transformation during mechanical loading and an α″ to β-phase transformation upon mechanical unloading.

In some embodiments, the alloy of the second aspect may comprise between 1 at. % and 8 at. % tin. In some embodiments, the alloy of the second aspect may comprise between 1 at. % and 10 at. % niobium. In some embodiments, the alloy of the second aspect may comprise between 0.5 at. % and 3 at. % iron. In some embodiments, the alloy of the second aspect may comprise up to 2 at. % oxygen. In some embodiments, the alloy of the second aspect may comprise up to 10 at. % zirconium.

In a third aspect, the present invention provides a method for producing the superelastic alloy of the first or second aspect of the present invention, the method comprising:
melting tin, niobium, iron, titanium and, optionally, zirconium, whereby a homogeneous alloy solution is produced;
cooling the alloy solution to produce an alloy ingot;
solution heat treating the alloy ingot by heating to a temperature at which a β-phase solid solution of the alloy is predominant; and
quenching the alloy, the as-quenched alloy retaining a metastable β-phase microstructure.

As will be described below, superelastic alloys produced in accordance with the method of the present invention have remarkably high recoverable strains, even without having been exposed to the further thermomechanical processing typically required in order to maximise an alloy's recoverable strain. Thus, the method of the present invention may provide an energy efficient method for producing superelastic alloys having an acceptable degree of superelasticity.

In some embodiments of the method of the present invention, the elements may be melted using vacuum-induction melting (VIM) or vacuum-arc melting (VAR). These techniques are relatively widely used to produce alloys and the necessary apparatus widely available.

In some embodiments, the method may comprise multiple melting and cooling (i.e. solidification) steps to produce the alloy ingot. Repeated melting/cooling steps ensure homogeneity of the elements throughout the alloy ingot.

In some embodiments, the alloy may be quenched by immersing in water (e.g. cold water), such being a highly effective way of retaining the β microstructure from the high temperature (above βₜᵣₐₙₛᵤₛ) β-phase field.

In some embodiments, the alloy ingot may be formed into an article (e.g. a wire or a sheet) before the solution heat treatment. During the formation process, atomic scale defects (e.g. dislocations, twins, etc.) could be induced, which might degrade the article's properties to some extent. Performing a short solution heat treatment at a temperature above βₜᵣₐₙₛᵤₛ followed by quenching right after the formation process might be used to “reset” and refine the microstructure and remove defects via recrystallization of R crystals.

In some embodiments, the tin, niobium, iron and zirconium (when present) may be provided in the form of elemental metals. In some embodiments, the titanium may be provided in the form of elemental titanium. In alternative embodiments (i.e. where the alloy includes oxygen), a small proportion of the titanium may be provided in the form of titanium oxide or lower grade titanium (e.g. “scrap” titanium), provided that the amount of oxygen is known (even if only roughly).

In some embodiments, the method may further comprise cold working (or other thermomechanical processing) the superelastic alloy in order to increase the recoverable strain of the alloy. The cold working may, in some embodiments, be followed by a short heat treatment (recrystallization or solution treatment) at a temperature above βₜᵣₐₙₛᵤₛ, followed by water quenching for reasons similar to those mentioned above (i.e. resetting the microstructure and removing potentially detrimental defects).

In a fourth aspect, the present invention provides a method for producing the superelastic alloy of the first or second aspect of the present invention, the method comprising:
melting tin, niobium, iron, titanium and, optionally, zirconium, whereby a homogeneous alloy solution is produced;
cooling the alloy solution to produce an alloy ingot;
solution heat treating the alloy ingot by heating to a temperature at which the alloy is a single β-phase solid solution (i.e. above the βₜᵣₐₙₛᵤₛ temperature); and
quenching the alloy, the as-quenched alloy retaining a metastable β-phase microstructure.

In a fifth aspect, the present invention provides the use of the alloy of the first or second aspect of the present invention for the manufacture of a shaped article.

In a sixth aspect, the present invention provides an orthodontic appliance comprising the alloy of the first or second aspect of the present invention. Examples of orthodontic appliances which may comprise or be formed of alloys in accordance with the present invention include dental archwires. Other superelasticity-demanding (resilience-demanding) applications include, for example, appliances for use in endodontic applications.

In a seventh aspect, the present invention provides a medical appliance comprising the alloy of the first or second aspect of the present invention. Examples of medical appliances which may comprise or be formed of alloys in accordance with the present invention include cardiovascular stents, heart valve frames, orthopaedic staples and single-use suture passers.

Other aspects, features and advantages of the present invention will be described below.

### BRIEF DESCRIPTION OF THE DRAWINGS

Embodiments of the present invention will be described in further detail below with reference to the accompanying drawings, in which:

FIG. 1 shows graphs depicting stress-strain curves as well as recoverable strain and residual strain values with respect to maximum applied strain for Ti-2.5Nb-2.5Fe-4Sn (at. %), Ti-2.5Nb-2.5Fe-5Sn (at. %) and Ti-2.5Nb-2.5Fe-6Sn (at. %) alloys (corresponding to the variable strain cyclic compression test);

FIG. 2 shows graphs depicting stress-strain curves as well as recoverable strain and residual strain values with respect to maximum applied strain for Ti-2.5Nb-2.5Fe-4Sn (at. %) and Ti-2.5Nb-2.5Fe-6Sn (at. %) alloys (corresponding to the constant strain cyclic compression test);

FIG. 3a is a SEM image of a micro-compression pillar fabricated via focused ion beam (FIB) to assess the superelastic responses of the Ti-2.5Nb-2.5Fe-5Sn (at. %) and Ti-2.5Nb-2.5Fe-6Sn (at. %) alloys along different crystallographic orientations, FIG. 3b is a graph depicting the stress strain curve associated with the <001>_{β} family of orientations of the Ti-2.5Nb-2.5Fe-5Sn alloy, exhibiting a recoverable strain of 7.3%, and FIG. 3c is a graph depicting the stress strain curve associated with the <001>_{β} family of orientations of the Ti-2.5Nb-2.5Fe-6Sn alloy, exhibiting a recoverable strain of 9%;

FIG. 4 is a graph showing the strength and elastic modulus of Ti-2.5Nb-2.5Fe-4Sn (at. %), Ti-2.5Nb-2.5Fe-5Sn (at. %) and Ti-2.5Nb-2.5Fe-6Sn (at. %, “the proposed alloys”) and that of the prior art Ti-6Al-4V, superelastic NiTi and Ti-16Nb-4.9Sn alloys;

FIG. 5 is a graph showing a comparison between the cell coverage ratio measured for a superelastic NiTi and the Ti-2.5Nb-2.5Fe-4Sn (at. %) alloy after 1, 3 and 7 days of incubation;

FIG. 6 shows the (a) Open Circuit Potential and (b) Potentiodynamic Polarization Tafel curves obtained for superelastic NiTi and the Ti-2.5Nb-2.5Fe-4Sn (at. %) alloy in PBS solution at 37° C.; and

FIG. 7 is a graph showing the density of the Ti-2.5Nb-2.5Fe-4Sn (at. %) alloy compared to that of other commonly used alloy systems in the medical and dental industries.

### DETAILED DESCRIPTION OF THE INVENTION

The overarching purpose of the present invention is to provide nickel-free titanium-based superelastic alloys that offer an alternative to and preferably an improvement over the (thus far) commercially unviable alternatives to NITINOL. It would be advantageous to provide economically producible nickel-free titanium based superelastic alloys consisting only of what are generally regarded as non-toxic and biocompatible elements and which exhibit a significant amount of superelasticity in articles for use in the biomedical and dental fields.

In one aspect, the present invention therefore provides a superelastic alloy comprising tin, in an amount of between 1 at. % and 8 at. %, niobium, in an amount of between 1 at. % and 10 at. % and iron, in an amount of between 0.5 at. % and 3 at. %. The alloy may also optionally comprise oxygen, in an amount of between 0 and 2 at. % and zirconium, in an amount of between 0 and 10 at. %. The balance of the alloy composition is titanium and unavoidable impurities. In the context of an alloy composition, unavoidable impurities may amount to up to about 1 at. % of the final composition.

In another aspect, the present invention provides a superelastic alloy consisting essentially of tin, niobium, iron, titanium and, optionally, oxygen and/or zirconium, the alloy having a metastable β-phase microstructure at human body temperature (i.e. about 37° C.) and exhibiting a β to α″-phase transformation during mechanical loading and an α″ to β-phase transformation upon mechanical unloading.

The alloys of the present invention are superelastic. Titanium-containing alloys exhibit superelasticity as a result of mechanical loading inducing a reversible phase transformation from their native metastable austenite phase (β-phase) into their martensite phase (α″ phase). A metastable β phase is required for superelasticity, but also the thermodynamic stability of the metastable β phase needs to be just sufficiently high to allow for a pure β phase to be retained when quenched from above the βₜᵣₐₙₛᵤₛ temperature but sufficiently low to allow for stress induced β to α″ forward and reverse transformations, during mechanical loading and unloading.

Provided that an alloy has the elemental composition disclosed herein, has a metastable β-phase microstructure at human body temperature and is able to undergo the defined phase transformation, the alloy would be expected to have superelastic properties. It is within the ability of a person skilled in the art, based on the teachings contained herein and using the techniques exemplified below, to determine an alloy composition falling within the scope of the second aspect of the present invention. Routine analysis using well established protocols and equipment such as X-ray diffraction (XRD), often in conjunction with Scanning and Transmission electron microscopy, as described below, can be used for determining the microstructure and constituent phases in an alloy. Differential scanning calorimetry (DSC) can be used to measure the specific temperatures associated with a phase transformation (i.e. martensitic transformation temperatures, in the present case). Further, superelasticity can be measured using well established protocols and equipment such as those described below, for example.

Alloys of the present invention have a metastable β-phase microstructure at human body temperature. It will be appreciated that the alloys do not necessarily have to have an absolutely single β phase microstructure, and that the presence of small amounts of α″ martensite phase or ω phase may not detrimentally affect the alloy's properties. Provided that the alloy has a dominant β-phase microstructure and has a stress required to induce martensitic transformation (σ_{SIM}) lower than the alloy's yield strength (which is a property inherent in a superelastic material), the inventors expect that it will be superelastic.

The phrase “metastable β-phase microstructure”, as used herein in the context of a superelastic alloy in accordance with the present invention, is therefore to be understood to mean a dominant metastable β-phase microstructure, where small amounts of other phases may be present, provided that they are not detrimental to the alloy's superelasticity.

The alloys of the present invention surprisingly and unexpectedly exhibit superelastic properties closer to those of superelastic NiTi alloys (albeit in an as homogenized-quenched state, as described below) than any other nickel-free titanium-based alloys of which the inventors are aware. Indeed, the maximum superelastic recoverable strain exhibited by alloys in accordance with the embodiments of the present invention described herein (in an as homogenized-quenched state) has been found to be as high as 4.6% (Ti-2.5Nb-2.5Fe-6Sn), which is significantly higher than what is believed to be the maximum superelastic recoverable strain exhibited by the closest prior art nickel-free superelastic β-Ti alloy (in an as homogenized-quenched state), which is only 3.75%.

The maximum superelastic recoverable strain of some NITINOL alloys is reported to be as high as 8%. Superelastic recoverable strain is, however, known to significantly improve by performing thermomechanical processing on the formed alloys, and the thermomechanical history of most NITINOL medical and dental products include some degree of cold working (which is a form of thermomechanical processing). Indeed, the inventors believe that the maximum superelastic recoverable strain of superelastic NiTi alloys (e.g. NITINOL) in an as homogenized-quenched state (i.e. where no mechanical processing of the alloy has yet been performed) is 6%.

At this time, thermomechanical processing has not been carried out on the alloys of the present invention. However, the inventors expect to observe a similar improvement in superelasticity for these alloys upon performance of such thermomechanical processing. As the alloys of the present invention exhibit superior superelasticity compared to the other alloys without having undergone any thermomechanical processing, it is expected that they will achieve even greater superelasticity after such processing.

The alloys of the present invention also consist of elements known to be more biocompatible than that of many existing titanium-based superelastic alloys such as NITINOL and the more recently developed nickel-free alternatives to NITINOL.

Additionally, the alloys of the present invention contain relatively low amounts of relatively expensive alloying elements (Nb), whilst taking advantage of economically effective and more readily available elements (Fe) to impart the beneficial properties described herein. As a result, the alloys of the present invention are expected to be cheaper to produce than many other superelastic alloys. Indeed, a cost analysis performed by the inventors, comparing alloys of the present invention with other, recently developed Ni-free superelastic alloys suggests that the average cost of the proposed alloys would be approximately a third. The primary reason for this is the relatively low content or absence of costly elements such as Nb and Ta, compared to the existing alloys.

Furthermore, the relatively low content of elements with a relatively high melting point (i.e. Nb) in the alloys of the present invention can help to significantly simplify the manufacturing process. For example, manufacturing routes such as conventional casting, powder metallurgy and additive manufacturing may be used to produce the alloys of the present invention. Further, some significant and common manufacturing issues (e.g. unmelted particles, inhomogeneity and evaporation of low melting point elements and melt anomalies) may not be relevant to the alloys of the present invention, due to their relatively low content of high melting point elements.

The superelastic alloys of the present invention include tin, niobium, iron, titanium and, optionally, oxygen and/or zirconium. The relative proportions of each of these elements in the alloys are interrelated, with the resultant alloy composition falling within the scope of the present invention provided that it has properties consistent with superelasticity, namely a metastable β-phase microstructure at human body temperature, a stress required to induce martensitic transformation (σ_{SIM}) lower than the alloy's yield strength and sufficient β-phase stability to allow for reverse (α″ to β) transformation upon mechanical unloading. Alloys having the composition defined by the first aspect of the present invention are all expected to be superelastic.

The superelastic alloys of the present invention comprise tin. The tin may be present in the alloy in an amount of between about 1 at. % and about 8 at. %. Tin helps to suppress undesired ω precipitation during the quenching process and also assists with fine-tuning the stability of the α″ martensite phase, thereby contributing to superelastic behaviour. The presence of tin in the alloy results in a relatively small amount of ω phase and can lead to complete suppression of α″ and ultimately better superelasticity. A sufficient amount of tin keeps the ω fraction low enough to not deteriorate the superelasticity of the alloys and high enough to contribute to precipitation strengthening of the alloys. Decreasing the Sn content would reduce the recoverable strain but should not diminish the superelastic properties, as long as the other elements are adjusted to compensate for the reduction in Sn content.

The amount of tin in the alloy will depend on factors such as the amounts of the other elements in the alloy and the alloy's intended purpose. If the amount of tin is increased above about 8 at. %, then undesired intermetallic phases may be promoted (e.g. Ti3-Sn intermetallics), leading to deterioration of superelasticity and other properties. The β phase may also become over-stabilised, leading to deterioration of superelasticity. If the amount of tin in the alloy is decreased below about 1 at. %, then the α″ martensite phase may become over-stabilised, leading to deterioration of superelasticity. Undesired solid-solution phases such as the ω phase may be promoted, leading to deterioration of superelasticity and other properties. The cost of the alloy may also increase, and its manufacturability negatively affected, due to the necessary increase in Nb content for compensating the decrease in Sn content. Increasing the Sn content to above 3-4 at. % may help to achieve the desired β-phase stability and decrease the fraction of the undesired athermal omega phase (that is also formed during quenching). A high proportion of omega phase is detrimental to superelasticity, although the effect of a relatively small fraction of omega phase on superelasticity is still unclear. In the embodiments described below, the addition of Sn above 3% led to significant reduction in omega phase fraction.

The alloy may, for example, comprise between about 4-6 at. % tin, between about 1-3 at. % tin, between about 3-5 at. % tin or between about 5-8 at. % tin. The alloy may, for example, comprise about 1, 1.5, 2, 2.5, 3, 3.5, 4, 4.5, 5, 5.5, 6, 6.5, 7, 7.5 or 8 at. % tin.

The superelastic alloys of the present invention also comprise niobium. The niobium may be present in the alloy in an amount of between about 1 at. % and about 10 at. %. The niobium helps to stabilise the β phase of the alloy during quenching and upon release of an applied load, thereby contributing to superelastic behaviour. The niobium also helps to suppress the α″ martensite phase and supresses the omega phase (to a lower extent compared to Sn). Niobium also reduces the β-transus temperature, once the components of the alloy have been homogenised, thereby lowering the temperature required for heat treatment and leading to lower processing costs.

The amount of niobium in the alloy will depend on factors such as the amounts of the other elements in the alloy and the alloy's intended purpose. The transformation strain of the resultant alloy will decrease with increasing Nb content, meaning that it is generally beneficial to keep the Nb content as low as possible in order to achieve the highest recoverable strain. Furthermore, niobium is relatively expensive and, due to its relatively high melting point, increases the complexity of alloy manufacture (making it more difficult to melt and cast). If the amount of niobium is increased above about 10 at. %, then the cost of the alloy increases disproportionately to the increase in superelasticity and, due to its high melting point, the alloy becomes more difficult to produce. However, as excessive Nb is not as detrimental as excessive Fe or Sn to the resultant alloy, the inventors note that increasing the Nb content up to about 10 at. % might further enhance the superelasticity of the alloys (while keeping the cost still lower than other Ni-free alloys).

If the amount of niobium in the alloy is decreased below about 1 at. %, then the α″ martensite phase may become over-stabilised, leading to deterioration of superelasticity and the Sn may lose its capacity to suppress the ω and α″ phases. Furthermore, Fe and Sn may have to increase excessively to compensate for the excessive reduction in Nb, leading to issues associated with excessive Sn or Fe.

The alloy may, for example, comprise between about 1-4 at. % niobium, between about 2-4 at. % niobium, between about 3-6 at. % niobium, between about 4-7 at. % niobium, between about 5-8 at. % niobium, between about 6-9 at. % niobium, between about 7-10 at. % niobium or between about 8-10 at. % niobium. The alloy may, for example, comprise about 1, 1.5, 2, 2.5, 3, 3.5, 4, 4.5, 5, 5.5, 6, 6.5, 7, 7.5, 8, 8.5, 9, 9.5 or 10 at. % niobium.

The superelastic alloys of the present invention also comprise iron. The iron may be present in the alloy in an amount of between about 0.5 and about 3 at. %. The iron also helps to stabilise the β phase of the alloy during quenching and upon release of an applied load, thereby contributing to superelastic behaviour. The iron also suppresses the α″ martensite phase and very slightly suppresses the ω phase. The iron can also significantly increase the yield strength of the resultant alloys via solid solution strengthening. The increase in yield strength of alloys with Fe addition results in larger gaps between the σ_{SIM} and yield strength, which can lead to enhancement of superelasticity.

The amount of iron in the alloy will depend on factors such as the amounts of the other elements in the alloy and the alloy's intended purpose. If the amount of iron is increased above about 3 at. %, then undesired intermetallic phases will be promoted, leading to deterioration of superelasticity and other properties. Furthermore, the β phase may become over-stabilised, leading to deterioration of superelasticity. If the amount of iron in the alloy is decreased below about 0.5 at. %, then the α″ martensite phase may become over-stabilised, leading to deterioration of superelasticity and undesired solid-solution phases (such as the ω phase) may be promoted, leading to deterioration of superelasticity and other properties. Furthermore, the cost of the alloy may increase, and its manufacturability be negatively affected, due to the necessity to increase Nb content in order to compensate for the decrease in Fe content.

Existing nickel-free superelastic alloys require a relatively large amount of noble alloying elements such as Nb in order to achieve the necessary β phase stability and hence superelasticity, and this is the primary reason for the excessively high cost of those alloys. The inventors discovered, however, that a desired β phase stability is surprisingly and unexpectedly still achievable if (expensive) Nb is replaced with (cheaper and more readily available) Fe. The inventors discovered therefore that the Nb content of an alloy could be significantly reduced (and hence the alloy become cheaper) by replacing it with Fe, and that this surprisingly resulted in an alloy having a superelastic recoverable strain significantly greater than that of any existing nickel-free superelastic alloy (with the same thermomechanical history—i.e. as-homogenised-quenched). Another benefit of decreasing the relative proportion of Nb in the alloy found by the inventors is that alloys containing relatively less Nb are easier to manufacture, as Nb's high melting point can cause the manufacturing issues described above.

The alloy may, for example, comprise between about 2-3 at. % iron, between about 1-2 at. % iron or between about 1-3 at. % iron. The alloy may, for example, comprise about 0.5, 1, 1.5, 2, 2.5, or 3 at. % iron.

The superelastic alloys of the present invention may also optionally comprise oxygen. Oxygen, in relatively small proportions, has been shown in the literature to act like a beta stabiliser (similar to Sn, Fe, Nb) and can beneficially increase the yield strength significantly, leading to a larger difference between the yield strength and the stress to induce martensitic transformation. This would be expected to enhance an alloy's superelasticity.

When present, the oxygen may be included in the alloy in an amount of between about 0-2 at. %. The inventors expect that oxygen may also help to suppress undesired ω precipitation during the quenching process and may also assist with fine-tuning the stability of the α″ martensite phase, thereby contributing to superelastic behaviour. Oxygen can also significantly increase the yield strength of alloys (more than Fe) and have an even stronger effect on suppressing the α″ martensite phase and promotion of β phase (the inventors note that these effects of oxygen are likely to be highly dependent on the presence and amount of other alloying elements). Increasing the yield strength of an alloy and increasing the gap between the σ_{SIM} and yield strength should, in principle, lead to better superelasticity.

If present, the amount of oxygen should be less than about 2 at. %, as higher proportions of oxygen in the alloy might result in a significant reduction in ductility and ultimate embrittlement of the alloys, which are undesirable properties of articles such as orthodontic archwires or implants, for example. Increasing the oxygen content above about 2 at. % may also suppress the β phase and promote the “opposing” low temperature a phase, which would be detrimental to the resultant superelasticity of the alloy.

If oxygen is present, the alloy may, for example, comprise between about 0.5-1.5 at. % oxygen. The alloy may, for example, comprise about 0.5, 1, 1.5 or 2 at. % oxygen.

Advantageously, small amounts of titanium oxide (e.g. in the form of a powder) or “scrap” titanium might be used (in addition to “pure” titanium, as the amount of oxygen in the alloy is very small compared to titanium) as a source of both titanium and oxygen for oxygen containing alloys, which might help to even further reduce the overall cost of the alloy.

For a more precise control of oxygen content in such embodiments, it may be better to use titanium oxide. However, scrap (low grade) titanium, if the oxygen and other impurities are within acceptable limits, might be able to be used as a source for both oxygen and titanium of the alloys. This might not result in as precise control of the alloy's oxygen content, but would likely be significantly cheaper.

In one embodiment, for example, high purity titanium may be used as the source for most of the titanium in the alloy, with oxygen being added in the form of Ti oxide, which will add the small remaining titanium content as well (TiO₂). Such embodiments enable a better control over oxygen content as well as other impurities, but would not be as cheap as the following embodiment.

In another embodiment, scrap or low grade titanium with the required (e.g. about 1 at. %) amount of oxygen may be used as the source for the total titanium and oxygen content of the alloys. Such embodiments would be cheaper, but would not enable as precise a control over the amount of oxygen, nor that of other impurities. Both of these embodiments should lead to a reduction in cost, however, with the latter embodiment having a more significant cost reduction effect compared to the former.

The superelastic alloys of the present invention may also optionally comprise zirconium. Zr is expected to impart similar properties to the resultant alloy to Sn, although a higher amount of Zr than Sn is likely to be required to achieve the same J-phase stability (due to a smaller effect on decreasing the martensitic transformation temperatures). Zr and Sn were both classified as neutral elements, however, recent studies have indicated that these elements can also act as Beta stabilisers when other Beta stabilisers (e.g. Nb and Fe) are present in the alloy.

The Zr may be present in the alloy in an amount of between about 0-10 at. %. The inventors expect that Zr may be beneficial in that it would keep the Sn content in moderate concentration and may further suppress the ω phase and also increase β stability. The Zr might also enhance the strength of the resultant alloys through solid solution strengthening of Zr. In compositions which contain relatively low amounts of Fe and/or Nb, the β stabilisation effect of Sn is low (the β stabilisation or ω/α″ suppression of Sn, and also Zr, depends on the type and amount of other alloying elements). Therefore, as the Sn content cannot be excessive (as explained above), Zr can be added to further suppress the ω and α″ phases.

If present, the amount of zirconium should be less than about 10 at. %, as higher proportions of zirconium in the alloy might over stabilise the β phase and thus deteriorate superelastic properties. The inventors believe that too much Zr might result in the formation of undesired Zr—Fe and/or Zr—Sn intermetallic phases, which phases could have an effect on the stability of the different phases (e.g. β or α″) and hence deteriorate the resultant alloy's superelastic properties.

If zirconium is present, the alloy may, for example, comprise between about 1-3 at. % zirconium, between about 4-6 at. % zirconium, between about 5-8 at. % zirconium, between about 6-9 at. % zirconium or between about 7-10 at. % zirconium. The alloy may, for example, comprise about 1, 2, 3, 4, 5, 6, 7, 8, 9 or 10 at. % zirconium.

In the specific embodiments described in further detail below, the nickel-free titanium-based alloy comprises 4, 5 or 6 at. % tin, 2.5 at. % niobium, 2.5 at. % iron and the balance titanium and unavoidable impurities. Such alloys are referred to herein as Ti-2.5Nb-2.5Fe-4Sn (at. %), Ti-2.5Nb-2.5Fe-5Sn (at. %) and Ti-2.5Nb-2.5Fe-6Sn (at. %), respectively.

The present invention also provides a method for producing the novel superelastic alloys described herein, the method comprising:
melting (in an inert environment) tin, niobium, iron, titanium and, optionally, zirconium whereby a homogeneous alloy solution is produced;
cooling the alloy solution to produce an alloy ingot;
solution heat treating the alloy ingot by heating (in an inert environment) the ingot to a temperature at which a β-phase solid solution of the alloy is predominant; and
quenching the alloy, the as-quenched alloy retaining a metastable β-phase microstructure (i.e. a dominant β-phase microstructure, as described above).

The alloys can be fabricated using pure titanium, niobium, tin and iron (and optionally zirconium) elements in the form of rods, turnings, foil, sponge and powder or other suitable forms. As noted above, titanium oxide in the form of powder may be used for oxygen-containing alloys.

The pure elements are weighted to ensure the correct relative atomic composition of the resultant alloy. The elements are then melted in an inert atmosphere or a vacuum using any suitable melting and solidification-based alloy ingot fabrication method such as vacuum-induction melting (VIM) or vacuum-arc melting (VAR). Typically, the method would include multiple melting and cooling steps to produce the alloy ingot, which would ensure macro-homogeneity, before it is allowed to solidify and cool down to room temperature inside the inert chamber.

The alloy ingots are solution heat treated by heating to a temperature at or above the βₜᵣₐₙₛᵤₛ temperature of the alloy composition, whereupon a β-phase solid solution of the alloy is predominant. Once the alloy ingot is heated to a temperature above the βₜᵣₐₙₛᵤₛ temperature, the alloy consists essentially of a single β-phase solid solution. The temperature can be raised above the βₜᵣₐₙₛᵤₛ temperature up to the solidus temperature (above which the liquid phase starts to appear), but there may be no benefit to doing so and the energy cost of such heating detrimental to the overall cost of production of the alloy. In some aspects, the alloy ingot is solution heat treated by heating to a temperature at which the alloy comprises a single β-phase solid solution (i.e. above the βₜᵣₐₙₛᵤₛ temperature).

The βₜᵣₐₙₛᵤₛ temperature is dependent on the composition of the alloy, and can be determined by a person skilled in the art using routine trials and experimentation. The βₜᵣₐₙₛᵤₛ temperature of an alloy can, for example, be theoretically calculated using thermodynamics simulation software such as the CompuTherm database commercially available from PANDAT™. The most common method for measuring the βₜᵣₐₙₛᵤₛ temperature is by using a Differential Scanning Calorimetry (DSC) device which measures the energy flow rate in or out of the sample (endothermic and exothermic reactions) while heating or cooling the sample to different temperatures.

In the case of the specific alloys described herein, the βₜᵣₐₙₛᵤₛ temperature is likely to be about 650° C. (923° K), although the inventors have not yet measured this and therefore, in the experiments described below, chose a heat treatment temperature of 1000° C., which they were confident would be above the alloys' βₜᵣₐₙₛᵤₛ temperatures.

In some embodiments, the alloy ingot may be formed into a product or an article before it is solution heat treated. For example, the ingot may be drawn into a wire before the solution heat treatment step, if the resultant product is for use as a superelastic dental archwire. Indeed, almost all NITINOL applications involve some form of solution heat treatment.

The inventors envisage being able to use at least two solution heat treatments. One is homogenisation, which may (but does not have to) be performed before wire drawing and right after fabricating the ingots. Homogenisation is a similar heat treatment to solution treatment, but the ingots do not necessarily have to be quenched (they can be slow cooled) afterwards and the purpose is not to retain the β phase but to just simply homogenize the ingots. It occurs at above β-transus.

As described above, the maximum recoverable strain of a superelastic alloy in its as-quenched state can generally be increased by thermomechanical processing (e.g. by cold working). Accordingly, the method of the present invention would therefore typically further comprise cold working the superelastic alloy, whereby a recoverable strain of the thermomechanically processed/cold worked alloy is increased.

An additional heat treatment (i.e. in addition to and subsequent to the solution heat treatment) is recrystallization treatment, which may (but does not have to) occur after wire drawing (or any other uni-directional mechanical processing or cold working) at a similar temperature as homogenisation heat treatment (i.e. above β-transus) but for a much shorter time. This heating is followed by quenching into cold water (e.g. ice-water) in order to recrystallise the β crystals after deformation (wire drawing), which resets and refines the microstructure. This is also believed to result in crystallographic texture which may significantly enhance the superelasticity.

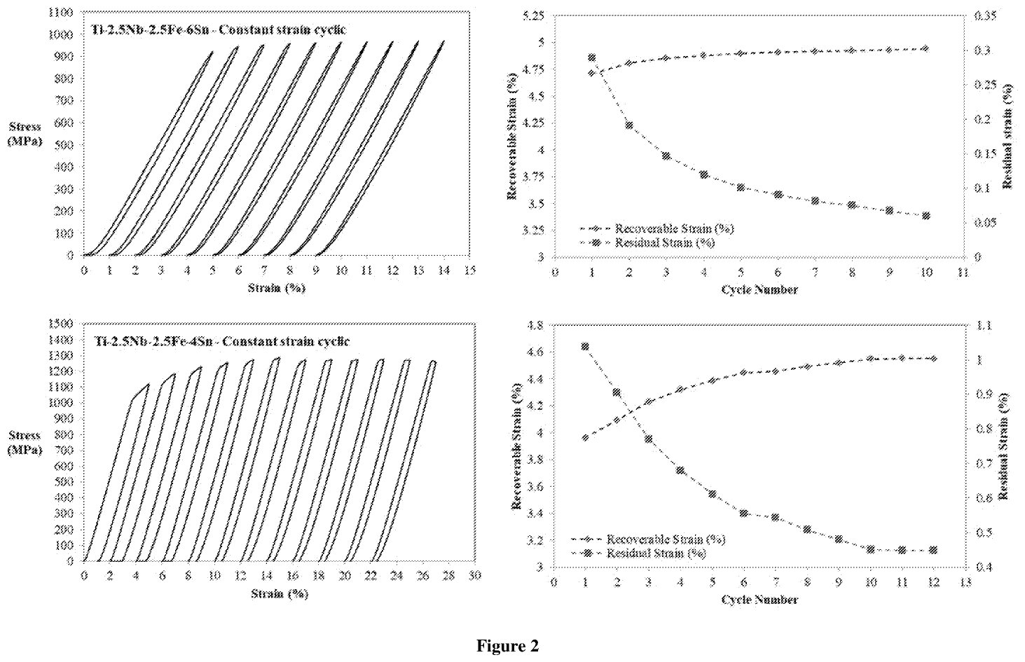

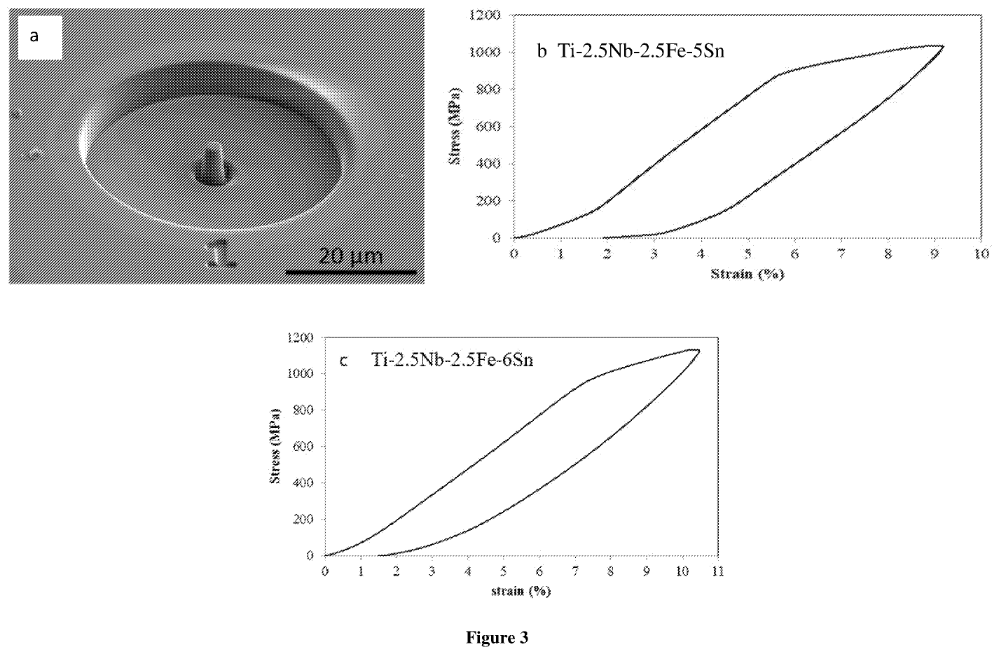

As partially demonstrated in the Examples set out below (referring to the description of FIGS. 2 and 3), thermomechanical processing of alloys in accordance with embodiments of the present invention could be performed in two ways, both of which should enhance the alloy's recoverable strain. In the first method, the alloys undergo a slight cold working (or work hardening) without any further heat treatment. The type of cold working in this method does not necessarily need to be a unidirectional one (e.g. wire drawing or rolling). The second method involves severe unidirectional cold working (such as severe rolling or wire drawing), followed by a recrystallization heat treatment which includes heating the cold worked ingot to above its βₜᵣₐₙₛᵤₛ in an inert environment, holding it at that temperature for a short period of time (could be even as short as 5 minutes or even shorter) and quenching it into cold water or ice-water to retain the β phase. The experimental results described below lead the inventors to believe that the second method could result in alloys having significantly higher recoverable strain.

In the embodiments described below where the alloys being produced are Ti-2.5Nb-2.5Fe-4Sn (at. %), Ti-2.5Nb-2.5Fe-5Sn (at. %) and Ti-2.5Nb-2.5Fe-6Sn (at. %), the alloy ingots were solution treated at a temperature between 1123° K-1273° K in an inert environment for time period range of 1 minute to 2 hours (e.g. 15 min, 30 min, 45 min, 1 hour, 1 hr 15 min, 1 hr 30 min, 1 hr 45 min or 2 hours) followed by water or ice-water quenching. The primary point of concern is retaining the high temperature R-phase upon quenching. Once the β phase is retained, due to the nature of the designed composition of the alloys, it will be mechanically unstable at room temperature. This results in reversible transformation of the retained β phase to α″ martensite phase upon mechanical loading and unloading. This stress induced transformation accommodates a significant amount of strain during mechanical loading. The reversibility of the transformation ensures full recovery of the amount of strain accommodated by the transformation upon unloading, resulting in a significant amount of recoverable strain and a small amount of permanent set at high applied strain values.

As will be described in further detail below, such alloys exhibit significant superelasticity even without having undergone significant thermomechanical processing. The processing consists only of a relatively short time homogenisation heat treatment above β-transus (800° C.-1000° C.) followed by water quenching and involves no costly mechanical processing (rolling, forging etc.) applied to the ingot after fabrication. As described above, this amount of processing, which has achieved the significant degree of superelasticity, is significantly lower than the other superelastic Ni-free Ti-based alloys in the literature. The superelastic recoverable strain exhibited by the alloys is also significantly higher than the other superelastic Ni-free Ti-based alloys in the literature that have undergone the same minimal thermo-mechanical processing.

Most (if not all) of the Ni-free superelastic Ti-based alloys reported in the literature are tested after undergoing some kind of severe plastic deformation such as rolling or forging followed by heat treatment and quenching. This means that the common processing route for the other Ni-free Ti-based superelastic alloys based on the literature is:
1. Fabrication (VAR or VIM)
2. Heat treatment+quenching
3. Mechanical processing (rolling etc.)
4. Heat treatment+quenching

Some of the alloys reported in literature also have undergone ageing heat treatments after step 4.

However, the alloy ingots in accordance with embodiments of the present invention (as described below) exhibit high recoverable strain only after undergoing steps 1 and 2. On an industrial scale, such extra heat treatments or necessary mechanical processing (i.e. steps 3 and 4) can be costly and may also limit the kind of products which these alloys can be turned into (for example, once the ingot is rolled the use is restricted to applications requiring a plate). For example, it would be unlikely that 3D printed parts made out of the Ni-free superelastic titanium alloys in the literature could exhibit a commercially relevant degree of superelasticity in an as-printed state, as it is not possible to mechanically process (e.g. roll) 3D printed parts without affecting their shape. Alloys in accordance with the present invention, however, already having a relatively high recoverable strain, may not need to undergo further mechanical processing and might be able to be used in their as-printed state (thereby making the alloys compatible with 3D printing).

Thus, whilst it may be beneficial to perform the extra thermomechanical processing descried above on the alloys of the present invention, it is not absolutely necessary because of the relatively high recoverable strains of the as-quenched alloys of the present invention.

The alloys of the present invention have a number of practical applications consistent with those of other superelastic materials, but with a significantly reduced toxicity and with a superelasticity that is potentially comparable to that of the best available product in the market (i.e. NITINOL).

In other aspects therefore, the present invention provides for the use of the nickel-free titanium-based superelastic alloys described herein for the manufacture of a shaped article. Also provided are dental (e.g. orthodontic and endodontic) and medical/biomedical appliances comprising the nickel-free titanium-based superelastic alloys described herein.

The alloys described herein can be applied to a wide range of applications, primarily in biomedical and dental fields. Some examples of such applications include those listed below.

### Medical Applications

Stents
Amplatzer/atrial septal occlusion devices
Simon (vena cava) filter
Orthopaedic Staples
Catheters
Tools for laparoscopy
   Variable curvature dissecting spatula (laparoscopic surgery)
   Clamps
   c Retractors
   Anastomotic rings
   Inguinal/umbilical hernia repair mesh
   etc.
Suture passing systems
Aortic sizer
Retrieval baskets
Guidewires/Catheters
Kidney stone extractor
Ablation devices
Surgical grippers
Deflectable graspers
Deflectable puncture needle
Harington rods
Frames for glasses

### Dental Applications

Orthodontic arch-wires
Endodontics
   Rotary and hand files
   Finger spreaders
   Crown and denture reconstructions

It will be appreciated that the alloys described herein may also find application in fields other than biomedical and dental. For example, they may be useful in construction, automotive, aerospace, defence, telecommunications, water/heating, clothing, sports and leisure fields.

### Examples

Specific alloys in accordance with embodiments of the present invention will now be described. Three alloys in accordance with specific embodiments of the present invention were prepared using the method described below. The superelastic properties of those alloys were then determined, as were the other chemical and physical properties described below.

The inventors believe that the description provided below in the context of these specific alloys is generally applicable to all alloys falling within the scope of the present invention, and that routine trial and experimentation using the techniques described herein, or those known in the art, could be used to confirm this.

### Manufacture of the Alloys

The superelastic alloys Ti-2.5Nb-2.5Fe-4Sn (at. %), Ti-2.5Nb-2.5Fe-5Sn (at. %) and Ti-2.5Nb-2.5Fe-6Sn (at. %) were produced using the method set out below.

The alloys were fabricated using pure titanium, niobium, tin and iron elements in the form of titanium and iron rods, niobium turnings and tin foil, all with purities of above 99.9%. The inventors note that elemental sponges and powders could also be used. The pure elements were weighed to ensure the correct relative composition of the alloy, and then melted using a common non-consumable arc-melting technique in an inert environment. The ingots were remelted a few times to ensure macro-homogeneity and were allowed to solidify and cool down to room temperature inside the inert chamber. The ingots were then encapsulated in quartz tubes with a partial pressure of pure argon. The encapsulated alloy ingots were then solution treated at 1273° K for one hour in an inert environment, followed by ice-water quenching. The quenching was performed by breaking the quartz tubes containing the alloy ingots in ice water in order to avoid excessive oxidation.

### Measurement of the Superelastic Recoverable Strain Exhibited by the Alloys

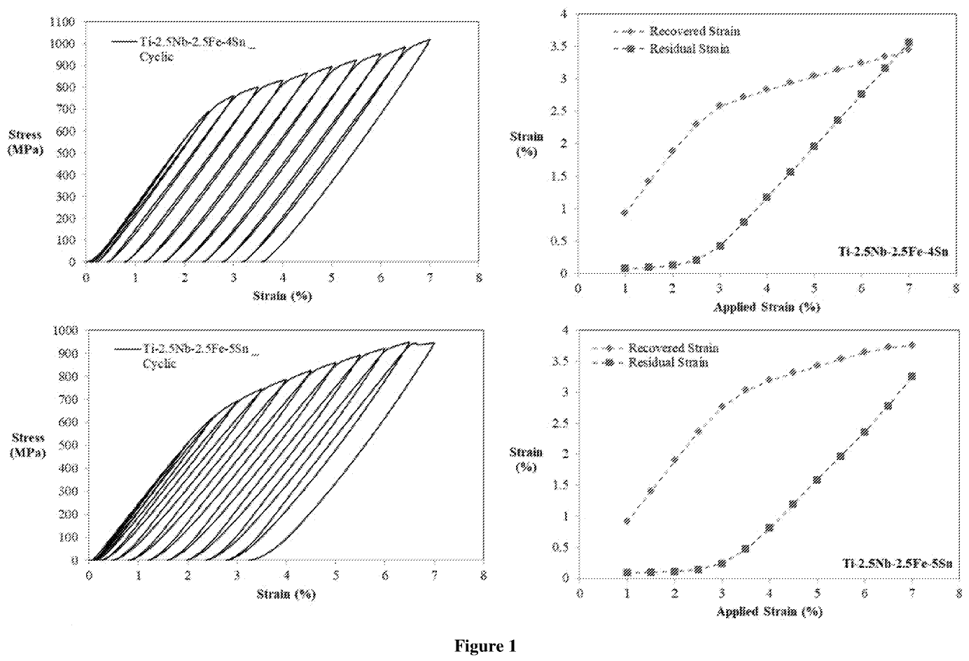

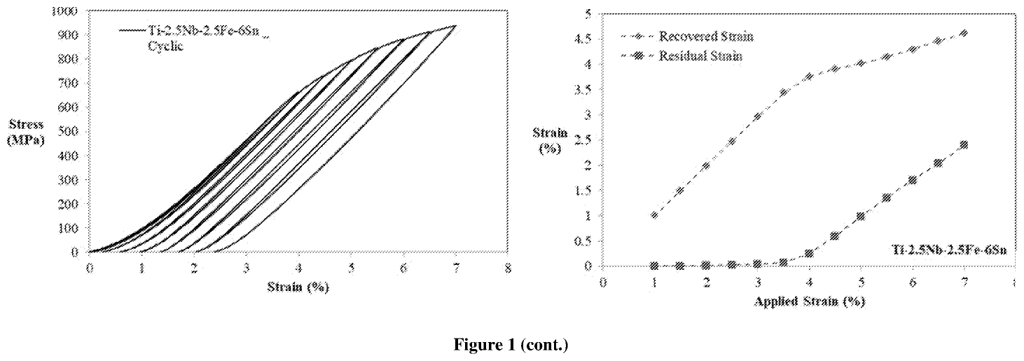

In order to quantify the amount of superelastic recoverable strain exhibited by the Ti-2.5Nb-2.5Fe-(4, 5 & 6) Sn alloys, a type of cyclic uniaxial compression test was performed on the alloys. During the course of this test, the alloy samples were loaded up to 1% strain, followed by unloading, until a force of 1N was reached. This was then repeated while increasing the applied strain by 0.5% after each cycle, and the test was terminated when a total applied strain of 7% was reached. The stress-strain curves thus obtained for the alloys are shown in FIG. 1.

This test allows for measuring the recoverable strain exhibited by the alloys with respect to different amounts of applied strain. The extreme work hardenability of the alloys encouraged the idea of performing a second type of cyclic uniaxial compression test to measure the maximum recoverable strain that the alloys are capable of exhibiting. This test included straining the already compressed specimens up to 5% (with respect to their new height, after the first cyclic test) until the absolute maximum recoverable strain is achieved and an asymptotic behaviour is observed. This test was performed on Ti-2.5Nb-2.5Fe-(4 & 6) Sn alloys. The results are shown in FIG. 2.

These cyclic compression tests showed two important aspects of the Ti-2.5Nb-2.5Fe-(4 & 6) Sn alloys. Firstly, they showed that the alloys are capable of exhibiting a recoverable strain of 3.4-4.6% at an applied strain of 7% in their as-homogenised and as-quenched state (i.e. with no further thermomechanical processing). This amount of recoverable strain is believed to be the largest achieved amongst the nickel-free titanium-based superelastic alloys in the literature (having the same thermomechanical history).

The second cyclic test revealed a significant increase in the recoverable strain with respect to the cycle number. This is believed to be the combinational effect of the “training process”, as well as an increase in yield strength of the material by work hardening. This indicates that the superelastic recoverable strain of the Ti-2.5Nb-2.5Fe-(4 & 6) Sn alloys is enhanced as they are cyclically loaded and unloaded. The results (FIG. 2) indicate that the maximum recoverable strain exhibited by the alloys can reach up to 4.5% and 5% for the Ti-2.5Nb-2.5Fe-4Sn and Ti-2.5Nb-2.5Fe-6Sn alloys, respectively.

As described above, the yield strength of the alloys might be increased by common grain refinement techniques, including severe plastic deformation followed by short-term heat treatment and quenching. The type of techniques used for inducing severe plastic deformation is the determining factor for induction of crystallographic texture in the treated specimens. The uni-directional mechanical processing techniques such as rolling and wire drawing are more likely to induce crystallographic texture in the specimen. Hence, in this case, if techniques such as forging, pressing or cross rolling (which are not uni-directional in nature) are used, followed by short-term heat treatment and quenching, a recoverable strain of approximately 5% is expected to be exhibited by the alloys. For the case of uni-directional mechanical processing followed by short heat treatment and quenching which could result in crystallographic texture, even larger recoverable strain values can be expected.

### Preliminary Experiments to Ascertain the Effect of Crystallographic Texture

As noted above, the inventors expect that cold working or other thermomechanical processing of these alloys (i.e. in their as-homogenised-quenched state) will increase their maximum recoverable strain, perhaps even as high as that of the superelastic NiTi (e.g. NITINOL) alloys.

In this regard, one of the more common manufacturing routes for superelastic self-expandable stents made from superelastic NiTi alloys involves drawing the alloy ingots into wire, followed by heat treatment and quenching. This is then followed by winding the wire around a specific jig in order to create the stent skeleton. The nature of this thermomechanical treatment results in a certain strength of crystallographic texture which is known to enhance the superelastic properties of the resultant alloy.

Hence, an experiment was designed in order to investigate the amount of superelastic recoverable strain exhibited by the Ti-2.5Nb-2.5Fe-5Sn and Ti-2.5Nb-2.5Fe-6Sn alloys of the present invention upon the induction of crystallographic texture. As crystallographic texture refers to a certain orientation of the crystals in the alloy, the experiment's objective was to investigate the superelastic recoverable strain exhibited by the different crystal (grains) orientations. This is expected to indicate the degree of superelastic recoverable strain exhibited by the alloys of the present invention once they go through a similar stent fabrication method currently performed on superelastic NiTi alloys. The experiment is explained in the following paragraphs.

Fifteen distinct micro-compression pillars were fabricated on the surface of the Ti-2.5Nb-2.5Fe-5Sn and Ti-2.5Nb-2.5Fe-6Sn alloys. The pillars were divided into five groups of three where each group was located on an individual grain with distinct crystal orientation. FIG. 3a shows a Scanning Electron Microscopy (SEM) image of one of the micro-compression pillars. The orientation of the grains was then measured using Electron Back Scatter Diffraction (EBSD) to find out which grain exhibits the closest orientation to the crystallographic texture which is expected to be obtained after uni-directional deformation (rolling or wire drawing) followed by short-term heat treatment and quenching. Pillars were then cyclically compressed using a Hysitron nano-indentation instrument. A spherical indentation tip with a radius of 5 μm was used. The pillars were compressed up to 1% strain followed by unloading until 1N force was reached. The maximum applied strain was then incremented by 0.5% after each cycle until fracture. FIG. 3b shows the stress strain curve obtained consistently across all three pillars on the grain oriented along the desired direction of the Ti-2.5Nb-2.5Fe-5Sn alloy. As can be observed, an average recoverable strain of 7.3% was consistently achieved. FIG. 3c shows the stress strain curve obtained consistently across all three pillars on the grain oriented along the desired direction of the Ti-2.5Nb-2.5Fe-6Sn alloy. As can be observed, an average recoverable strain of 9% was consistently achieved.

This test indicates another important property of the alloys in accordance with the present invention, namely that if the same thermomechanical procedure commonly applied to the stent wires formed from superelastic NiTi alloys were to be performed on the present alloys, a recoverable strain of as much as 9% might be expected due to the recrystallization texture. This value is similar to that exhibited by the superelastic NiTi alloys in the form of stent wire. The common thermomechanical procedure applied onto the superelastic NiTi alloys in the industrial scale, as mentioned before, consists of unidirectional severe plastic deformation followed by short-term heat treatment and quenching. This is due to the recrystallization texture that is inherently induced by the nature of the thermomechanical process. This significant amount of recoverable strain is comparable with that of superelastic NiTi alloys under the same thermomechanical condition.

### Other Relevant Mechanical Properties

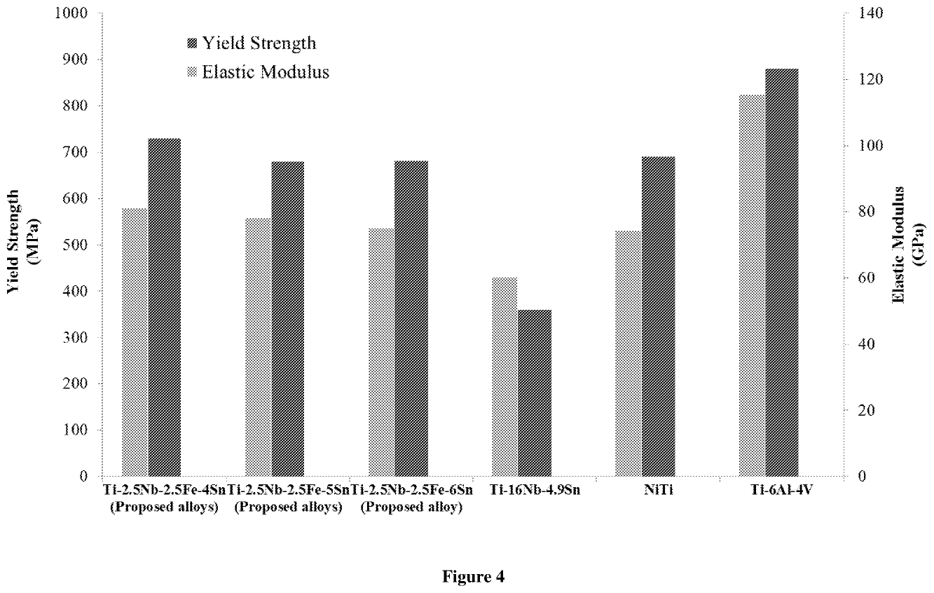

Other important mechanical properties for biomedical and dental superelasticity-demanding applications are the yield strength (which is directly proportional to hardness) and elastic modulus of the alloy. In this regard, FIG. 4 shows a comparison between these properties exhibited by the three Ti-2.5Nb-2.5Fe-(4, 5 & 6) Sn alloys of the present invention, as well as those of the prior art Ti-6Al-4V alloy, the superelastic NiTi alloy (NITINOL) and the nickel-free superelastic alloy Ti-16Nb-4.9Sn.

As is observed, the Ti-2.5Nb-2.5Fe-(4, 5 & 6) Sn alloys exhibit a similar elastic modulus and yield strength to that of the superelastic NiTi. This is particularly important as the higher strength is directly proportional to higher stability of the superelastic properties. This is because a higher strength results in a smaller amount of plastic deformation at specific stress and strain levels. On the other hand, higher strength could potentially indicate better fatigue properties at a specific stress amount compared to superelastic alloys with lower strength (such as Ti-16Nb-4.9Sn). Additionally, as hardness and strength are directly proportional, a higher strength could also correspond to higher hardness and ultimately higher resistance to wear. This is also important as one of the primary mechanisms of release of toxic Ni ions from the surface of NiTi implants is believed to be due to wear, leading to breakage of the passive oxide layer and ultimately exposure of the fresh bulk alloy to the corrosive body environment.

### Cytocompatibility of the Alloys

Cytocompatibility of the Ti-2.5Nb-2.5Fe-4Sn alloy was assessed through cell density and cell attachment investigations. The Ti-2.5Nb-2.5Fe-4Sn alloy was considered as representation of the proposed alloys, and was compared with the superelastic NiTi alloy Nitinol-55 (Ti-51Ni at. %). The Chinese Hamster Ovarian (CHO) cell line was considered for this investigation because it has been reported to be effective for cytotoxicity evaluation of biomaterials. The CHO cells used in this investigation were genetically modified to express a Green Fluorescent Protein (GFP) for easier confocal microscopy analysis. Pure raw materials (Ti, Nb, Fe, Sn and Ni) were used to fabricate ingots of both alloys via non-consumable arc-melting technique. The ingots were then encapsulated in quartz tubes under partial pressure of argon and placed in furnace at 1273 K for 2 hours for solution treatment. This was then followed by quenching the ingots into ice-water. An Electro-Discharge Machine (EDM) was then used to cut a total of nine square samples per composition (total of 18), each having a cross sectional area of 9 mm² and a thickness of 2 mm, from the solution-treated ingots. The square samples were then mechanically ground and sonicated in acetone and ethanol to obtain a clean surface free of discoloured oxidized layer. Both the top and bottom surfaces of the samples were sufficiently ground using a 600 grit (14 μm) SiC paper to ensure similar amount of roughness on all sample of both compositions.

All the samples were then sterilized by autoclaving at 393 K for 30 minutes prior to the cytocompatibility investigation. Cell density and cell attachment were evaluated via confocal and scanning electron microscopy, respectively. The samples of each composition were divided into three groups of three to be incubated for 1-day, 3-days and 7-days. The samples were then placed in 24-well plates and seeded with CHO cells in Alpha Modified Eagle Medium (α-MEM) which was supplemented with 10% feta bovine serum (FBS) and 1% Penicillin Streptomycin (P/S). The cell per well density was chosen to be 40000 cells per well.

The 24 well plates containing the alloy samples and cells were then incubated at 37° C. in 5% CO₂ atmosphere. Once the incubation time periods (1-day, 3 days and 7-days) corresponding to the particular samples reached completion, the cells were then rinsed with Phosphase Buffer Solution (PBS). The cells were then fixed using paraformaldehyde for 30 minutes. Confocal analysis was then performed on the samples using an Olympus N-STORM SuperResolution Confocal Microscope. After the confocal analysis the samples were then dehydrated using 50 vol. %, 70 vol. %, 90 vol. % and 95 vol. % ethanol solutions for 10 minutes each, followed by 100 vol. % ethanol solution for 15 minutes. The final step was repeated for 30 minutes and the samples were air dried for 15 hours. The samples were then coated with gold to enhance the conductivity required for Scanning Electron Microscopy (SEM). The SEM imaging of the cells was carried out using a FEI SCIOS dual-beam SEM instrument in order to evaluate the cell attachment.

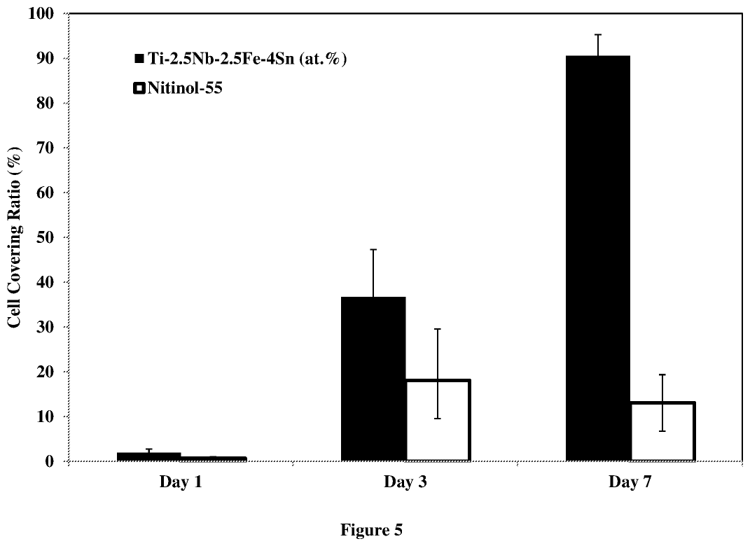

The confocal microscopy images obtained from the Nitinol-55 (NiTi) and Ti-2.5Nb-2.5Fe-4Sn (at. %) alloys with identical thermomechanical history, after 1 day, 3 days and 7 days of cell culture show that a higher cell density for the Ti-2.5Nb-2.5Fe-4Sn alloy can be clearly observed, even from the first day. The difference between the cell coverage on the alloys becomes more significant as the number of incubation days increases. This can be explained by the gradual corrosion and hence release of Ni ions from the surface of the Nitinol-55 samples. In order to quantify the cell density or cell coverage and compare the two alloys, multiple confocal images covering an area of 1 mm by 1 mm were taken from each sample (3 samples per time period). The images were then used to calculate the Cell Coverage Ratio (CCR), which is defined as the ratio between the area of the sample surface covered by alive cells divided by the total field of view of the images (1 mm by 1 mm). The CCR results are shown in FIG. 5.

As is observed, the CCR exhibited by the Ti-2.5Nb-2.5Fe-4Sn alloy is significantly higher than that of the Nitinol-55 after 3 and particularly 7 days. This is a clear indication of significantly lower toxicity associated with the proposed alloys compared to Nitinol-55.

The cell attachment was also qualitatively assessed via Scanning Electron Microscopy (SEM) imaging. In the case of CHO cells, elongated cell morphology is a clear indication of superior bioactivity, and this was observed (results not shown) for the Ti-2.5Nb-2.5Fe-4Sn alloy in comparison with the Nitinol-55. The cell morphology after 7 days on the surface of the proposed Ti-2.5Nb-2.5Fe-4Sn alloy was observed to be clearly elongated and the thick cell stacking network is evident indicating great bioactivity of the surface. The 7-day cell morphology of the Nitinol-55 alloy however, was clearly indicative of a large number of dead cells and significant toxicity caused by the toxic Ni-containing surface chemistry.

### Corrosion Resistance of the Alloys

Corrosion resistance of the Ti-2.5Nb-2.5Fe-4Sn alloy was also investigated and compared with that of Nitinol-55. One sample of Nitinol-55 and one of the Ti-2.5Nb-2.5Fe-4Sn alloy with a surface area of 3 mm by 3 mm were prepared and mounted in a non-conductive epoxy resin with a copper wire attached to the back of the samples. The samples were then ground to the same degree of 600 grit or 14 μm, followed by thorough sonication cleaning in acetone, ethanol and distilled water for 10 minutes. The samples were then air dried. The media chosen and prepared for this corrosion study was a Phosphate Buffered Saline (PBS) simulated body fluid solution. The solution consisted of 8 gl-1 of NaCl, 0.2 gl-1 of KCl, 2.9 gl-1 of Na₂HPO₄.12H₂O and 0.2 gl-1 of KH₂PO₄. The pH of the solution was adjusted to be 7.4 using HCl. The corrosion analysis was conducted using a potentiostat and by submerging the surface of samples into the fresh PBS solution at 37° C. A typical three electrode cell was used, with a platinum counter electrode (CE) and Ag—AgCl electrode as the reference electrode (RE).

The Open Circuit Potential (OCP) was performed for 24 hours for each sample, initiating from the moment the samples were in contact with the solution. Additionally, the potentiodynamic polarization analysis was carried out with respect to the OCP after one hour of immersion between −1V to +2V at a scanning rate of 0.667 mV/s.

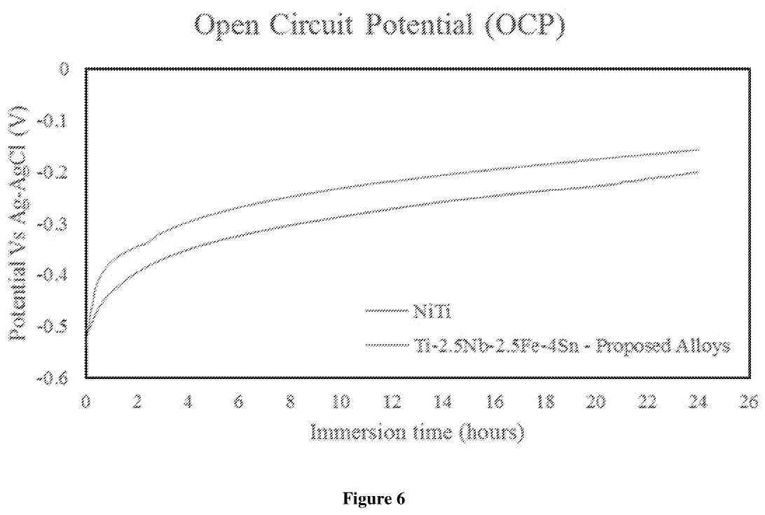

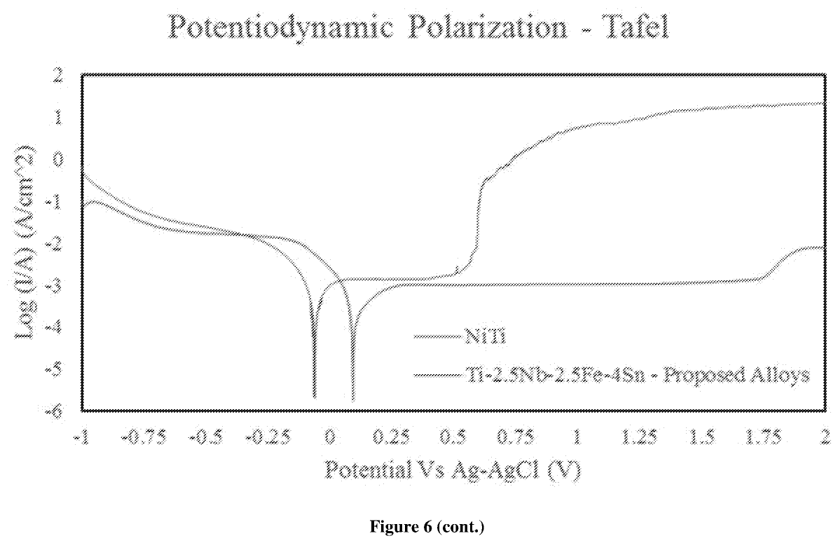

As FIG. 6 shows, the potential values corresponding to both Nitinol-55 and the Ti-2.5Nb-2.5Fe-4Sn alloy shift towards the positive direction. This is an indication of the formation of protective passive surface oxide films. The reduction in the slope of the OCP curves with respect to time indicates the progression of passivity of this layer.

The potentiodynamic polarization analysis was performed to gain a better understanding about the stability and continuity of the oxide film formation on the surface of both alloys. The Tafel curve clearly exhibits the active-passive region corresponding to both alloys. The corrosion potential values can be estimated from such regions as −0.062 V and 0.117 V for Nitinol-55 and Ti-2.5Nb-2.5Fe-4Sn alloys, respectively. Similarly, the Tafel analysis of the anodic and cathodic branches of the polarization curves was used to estimate the corrosion current density for both alloys. These values were found to be 0.049 and 0.031 pA/cm² for Nitinol-55 and Ti-2.5Nb-2.5Fe-4Sn, respectively.

The significantly wider passivation region exhibited by the Ti-2.5Nb-2.5Fe-4Sn alloy (0.1 to 1.75V) compared to that exhibited by Nitinol-55 (−0.07 to 0.45V) is clearly based on the Tafel curves shown in FIG. 6. Additionally, the Ti-2.5Nb-2.5Fe-4Sn alloy exhibits a constant current density up to 1.75V, while that value is as low as 0.45V for Nitinol-55. Furthermore, the rise in the current density after 1.45V exhibited by the Ti-2.5Nb-2.5Fe-4Sn alloy is followed by clear re-passivation at 1.94V up to 2V. This is while the increase in current density at 0.45V exhibited by Nitinol-55 is continued until the end of voltage spectrum (at 2V), indicating low tendency for passivation. Such observations indicate relatively greater resistance to localised corrosion exhibited by the Ti-2.5Nb-2.5Fe-4Sn alloy compared with Nitinol-55.

It is believed that this ultimately results in the formation of unstable oxide layers on the surfaces of superelastic NiTi alloys, leading to release of nickel into the body fluids. The mechanism which drives this phenomenon is generally known as corrosion pitting. In relevant experiments performed by the inventors (results not shown), the severity of the corrosion pitting on the surface of Nitinol-55 can be observed easily while no pitting was observed on the surface of the proposed alloy.

### Density

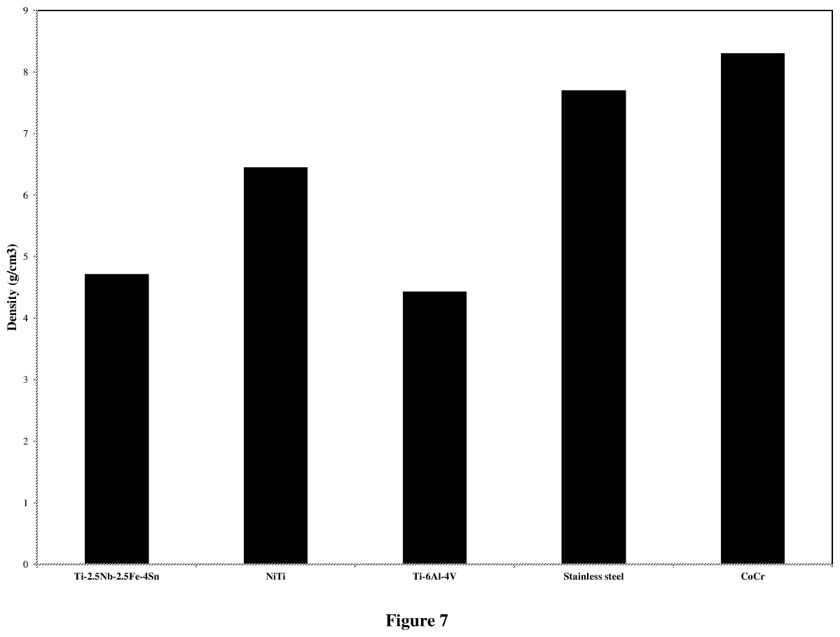

Additionally, the density of the Ti-2.5Nb-2.5Fe-4Sn and Nitinol-55 (referred to as “NiTi” in FIG. 7) alloys were measured via the Archimedes technique. The homogenised and quenched samples cut from arc melted ingots were used for this analysis. All surfaces of the samples were initially ground using standard 600 grit sand papers in order to remove any oxidised or contaminated surface layer. The samples were then sonicated in acetone and ethanol for 15 minutes (total of 30 minutes). This was then followed by placing the samples in an oven at 120° C. to ensure that the samples are completely dry. The dry mass of the samples was then measured using a high precision scale. The samples were then placed in a vacuum chamber to allow the air to escape from all possible pores and then submerged in water for 30 minutes allowing water to fill the pores within samples. The samples were then removed from the chamber while keeping them submerged in a smaller container. The submerged apparent mass of the samples was then measured using a suspension support from the scale. The samples were then removed from the water and were immediately placed onto the scale to measure their saturated mass. These values are shown in Table 1.

TABLE 1
Archimedes principle parameters measured
for Nitinol-55 and Ti—2.5Nb—2.5Fe—4Sn
alloys for density measurement.

| | Dry | Saturated | Submerged | Density |
|---|---|---|---|---|
| Alloys | Weight (g) | Weight (g) | Weight (g) | (g/cm³) |
| Nitinol-55 | 6.81 | 6.87 | 5.76 | 6.49 |
| Ti—2.5Nb—2.5Fe—4Sn | 4.71 | 4.76 | 3.77 | 4.71 |

Using these values, the density of the Ti-2.5Nb-2.5Fe-4Sn alloy was measured (see Table 1). The density of Nitinol-55 has been measured previously and been reported in the literature, however it was measured again here for validation and comparison purposes. The density of the Ti-2.5Nb-2.5Fe-4Sn alloy, Nitinol-55, Ti-6Al-4V wt. %, stainless steel and CoCr alloy (the densities of the stainless steel and CoCr alloys are from literature) are shown in FIG. 7. Nitinol-55, CoCr and Stainless steel are the most common materials used for stents, whilst Ti-6Al-4V is the most commonly used non-superelastic titanium based alloy in the medical sector.

As can be seen, the Ti-2.5Nb-2.5Fe-4Sn alloy has a lower density than Nitinol-55, the stainless steel and the CoCr alloy. The density is relevant to both the alloy's economics and its functionality. In terms of economics, a larger number of products, for example stents (having the same design and geometry or volume), can be fabricated for a given weight with a lower density material compared to that of a higher density material. Hence, one kilogram of the proposed alloys can lead to a greater number of products with the same volume compared to one kilogram of Nitinol-55, CoCr alloy or stainless steel. Further, the lower density of the alloy of the present invention suggests that lighter implants as well as dental and medical devices can be manufactured having similar properties to those of the (heavier) Nitinol-55, CoCr alloy or stainless steel.

Implants and devices made from the alloys in accordance with the present invention, as described herein, can thus be manufactured with an intentionally larger amount of material in order to match the weight of identical devices made out of superelastic NiTi alloys. This essentially means that articles made out of the present alloys and superelastic NiTi alloys would have the same weight, however, the articles made out of the proposed alloys will be stronger and will have better properties.

The inventors also note that the initial material cost per kilogram of the proposed alloys is similar to that of superelastic NiTi alloys and therefore, in light of the advantages of its relatively lower density, the alloys of the present invention may not only be more economical than the other Ni-free Ti based superelastic alloys, but also more economical than superelastic NiTi alloys.

As described herein, the present invention provides nickel-free titanium-based superelastic alloys. Embodiments of the present invention provide a number of advantages over existing titanium-based superelastic alloys and nickel-free titanium-based superelastic alloys, including:
Significantly lower amount of alloying elements than existing superelastic NiTi alloys and Ni-free Ti based superelastic alloys.
   Significantly higher ease of manufacturing, regardless of manufacturing process.
   Significantly lower material cost (as low as one third of the average cost of Ni-free superelastic titanium based alloys).
Significantly larger recoverable strain in as-homogenized-quenched state.
   No further thermomechanical processing is required for some superelasticity-demanding applications.
   After the application of thermomechanical processing, and thereby induced crystallographic texture, comparable superelastic properties to that of superelastic NiTi alloys (e.g. NITINOL) is expected.
The alloys would solve the nickel toxicity related issues associated with the use of superelastic NiTi alloys.

It will be understood to persons skilled in the art of the invention that many modifications may be made without departing from the spirit and scope of the invention. All such modifications are intended to fall within the scope of the following claims.

It is to be understood that any prior art publication referred to herein does not constitute an admission that the publication forms part of the common general knowledge in the art.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A superelastic alloy comprising:
tin, in an amount of between 1 at. % and 8 at. %;
niobium, in an amount of between 1 at. % and 10 at. %;
iron, in an amount of between 0.5 at. % and 3 at. %; and
optionally:
oxygen, in an amount of between 0 and 2 at. %; and
zirconium, in an amount of between 0 and 10 at. %,
the balance being titanium and unavoidable impurities.

2. The alloy of claim 1, comprising between 4-6 at. % tin.

3. The alloy of claim 1 or claim 2, comprising between 1-4 at. % niobium.

4. The alloy of any one of claims 1 to 3, comprising between 2-3 at. % iron.

5. The alloy of any one of claims 1 to 4, comprising between 0.5-1.5 at. % oxygen.

6. The alloy of any one of claims 1 to 5, comprising 4 at. % tin, 2.5 at. % niobium, 2.5 at. % iron and the balance titanium and unavoidable impurities.

7. The alloy of any one of claims 1 to 5, comprising 5 at. % tin, 2.5 at. % niobium, 2.5 at. % iron and the balance titanium and unavoidable impurities.

8. The alloy of any one of claims 1 to 5, comprising 6 at. % tin, 2.5 at. % niobium, 2.5 at. % iron and the balance titanium and unavoidable impurities.

9. A superelastic alloy consisting essentially of tin, niobium, iron, titanium and, optionally, either or both of oxygen and zirconium, the alloy having a metastable β-phase microstructure at human body temperature and exhibiting a β to α″-phase transformation during mechanical loading and an α″ to β-phase transformation upon mechanical unloading.

10. The alloy of claim 9, comprising between 1 at. % and 8 at. % tin.

11. The alloy of claim 9 or claim 10, comprising between 1 at. % and 10 at. % niobium.

12. The alloy of any one of claims 9 to 11, comprising between 0.5 at. % and 3 at. % iron.

13. The alloy of any one of claims 9 to 12, comprising up to 2 at. % oxygen.

14. The alloy of any one of claims 9 to 13, comprising up to 10 at. % zirconium.

15. A method for producing the superelastic alloy of any one of claims 1 to 14, the method comprising:
melting tin, niobium, iron, titanium and, optionally, zirconium whereby a homogeneous alloy solution is produced;
cooling the alloy solution to produce an alloy ingot;
solution heat treating the alloy ingot by heating to a temperature at which a β-phase solid solution of the alloy is predominant; and
quenching the alloy, the as-quenched alloy retaining a metastable β-phase microstructure.

16. The method of claim 15, wherein the elements are melted using vacuum-induction melting (VIM) or vacuum-arc melting (VAR).

17. The method of claim 15 or claim 16, comprising multiple melting and cooling steps to produce the alloy ingot.

18. The method of any one of claims 15 to 17, wherein the alloy is quenched by immersing in cold water.

19. The method of any one of claims 15 to 18, wherein alloy ingot is formed into an article before the solution heat treatment.

20. The method of any one of claims 15 to 19, further comprising cold working the superelastic alloy whereby a recoverable strain of the cold worked alloy is increased.

21. The use of the alloy of any one of claims 1 to 14 for the manufacture of a shaped article.

22. An orthodontic appliance comprising the alloy of any one of claims 1 to 14.

23. A medical appliance comprising the alloy of any one of claims 1 to 14.
